(19) United States
(12) Patent Application Publication
(10) Pub. No.: US 2014/0213998 A1
(43) Pub. Date: Jul. 31, 2014

(54) SYSTEM AND METHOD FOR ADMINISTERING INSULIN

(71) Applicant: GLUCOME LTD., YARKONA (IL)

(72) Inventors: DOV MORAN, KFAR SABA (IL); YIFTAH BEN AHARON, MOSHAV BAKOA (IL)

(21) Appl. No.: 14/166,957

(22) Filed: Jan. 29, 2014

Related U.S. Application Data
(60) Provisional application No. 61/758,287, filed on Jan. 30, 2013.

(51) Int. Cl. A61M 5/172

(52) U.S. Cl. CPC A61M 5/1723
USPC 604/504; 604/66

(57) ABSTRACT

A method of administering insulin to a user is provided. The method comprises providing a system comprising a glucometer configured to analyze a blood sample, a remote computing device separate from the glucometer, and an insulin pump separate from the remote computing device and glucometer, analyzing, by the glucometer, a blood sample from a user, and communicating the results to the remote computing device, determining, by the remote computing device and based on the results, an insulin dosage to be administered, communicating, by the remote computing device, a command to the insulin pump to administer a dose of insulin based on the determined dosage, and administering, by the insulin pump, the dose.

### CROSS-REFERENCE TO RELATED APPLICATIONS

This application claims the benefit of priority from U.S. Provisional Patent Application No. 61/758,287, filed Jan. 30, 2013, the contents of which are incorporated herein by reference in their entirety.

### FIELD OF THE INVENTION

The disclosure herein relates to the methods and systems for administering a dose of insulin to a user.

### BACKGROUND OF THE INVENTION

Diabetes is a metabolic disease characterized by high blood sugar, also called glucose, resulting from disruption in production of, or lack of proper response to, insulin, a hormone central to regulating carbohydrate and fat metabolism.

Diabetes can cause serious health complications including heart disease, blindness, kidney failure, and lower-extremity amputations. These complications may be avoided through effective and efficient balance of sugar levels. The insulin pump is one tool used to maintain an optimal balance of blood sugar by regulating the level of insulin in a user.

Typically, a user will measure his blood sugar, for example using a glucometer. Based on the measured blood sugar, he uses an insulin pump to administer an appropriate dose of insulin.

### SUMMARY OF THE INVENTION

According to one aspect of the presently disclosed subject matter, there is provided a method of administering insulin to a user. The method comprises providing a system comprising a glucometer configured to analyze a blood sample, a remote computing device separate from the glucometer, and an insulin pump separate from the remote computing device and glucometer; analyzing, by the glucometer, a blood sample from a user, and communicating the results to the remote computing device; determining, by the remote computing device and based on the results, an insulin dosage to be administered; communicating, by the remote computing device, a command to the insulin pump to administer a dose of insulin based on the determined dosage; and administering the dose by the insulin pump.

The determining may comprise calculating a dosage to be administered.

The determining may comprise retrieving dosage information from one or more tables preloaded on the remote computing device.

The remote computing device may be configured to automatically communicate the command.

The remote computing device may be configured to communicate the command upon confirmation by a user.

The communicating may further comprise transmitting one or more of error-detection and error-correction information.

The method may further comprise the system verifying that an intended command was received by the insulin pump.

The method may further comprise, prior to the administering, a user activating a mechanism on the insulin pump.

According to another aspect of the presently disclosed subject matter, there is provided a system configured to administer insulin to a user, the system comprising a glucometer configured to analyze a blood sample, a remote computing device separate from the glucometer, and an insulin pump separate from the remote computing device and glucometer, wherein the remote computing device is configured to receive communication from the glucometer regarding results of a blood analysis, determine, based on the results, an insulin dosage to be administered, and communicate a command to the insulin pump to administer a dose of insulin based on the determined dosage, the insulin pump being configured to administer the dose.

The remote computing device may be configured to determining the insulin dose by calculating a dosage to be administered.

The remote computing device may be configured to determining the insulin dose by retrieving dosage information from one or more tables preloaded on the remote computing device.

The remote computing device may be configured to automatically communicate the command.

The remote computing device may be configured to communicate the command upon confirmation by a user.

The remote computing device may be further configured to transmitting one or more of error-detection and error-correction information to the insulin pump.

The system may be configured to verify that an intended command was received by the insulin pump.

The insulin pump may be configured to administer the dose upon activation of a mechanism thereof.

According to a further aspect of the presently disclosed subject matter, there is provided a device configured to administer an insulin dose to a user, the device comprising a pump configured for the administering, a communications interface configured to receive instructions from a remote computing device, and a processor, the processor being configured to receive at least one instruction via the communications interface and operate the pump to administer an insulin dose based on the instruction.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1 is a schematic illustration of a system according to the presently disclosed subject matter;

FIG. 2 is a schematic illustration of a glucometer of the system illustrated in FIG. 1;

FIG. 3 is a schematic illustration of an insulin pump of the system illustrated in FIG. 1;

FIG. 4 is a schematic illustration of a remote computing device of the system illustrated in FIG. 1; and

FIG. 5 illustrates a method by which the system illustrated in FIG. 1 administers a dose of insulin.

### DETAILED DESCRIPTION OF THE INVENTION

As illustrated in FIG. 1, there is provided a system, which is generally indicated at 10, for measuring the glucose level of a user. The system 10 comprises a glucometer 12, an insulin pump 14, and a remote computing device 16.

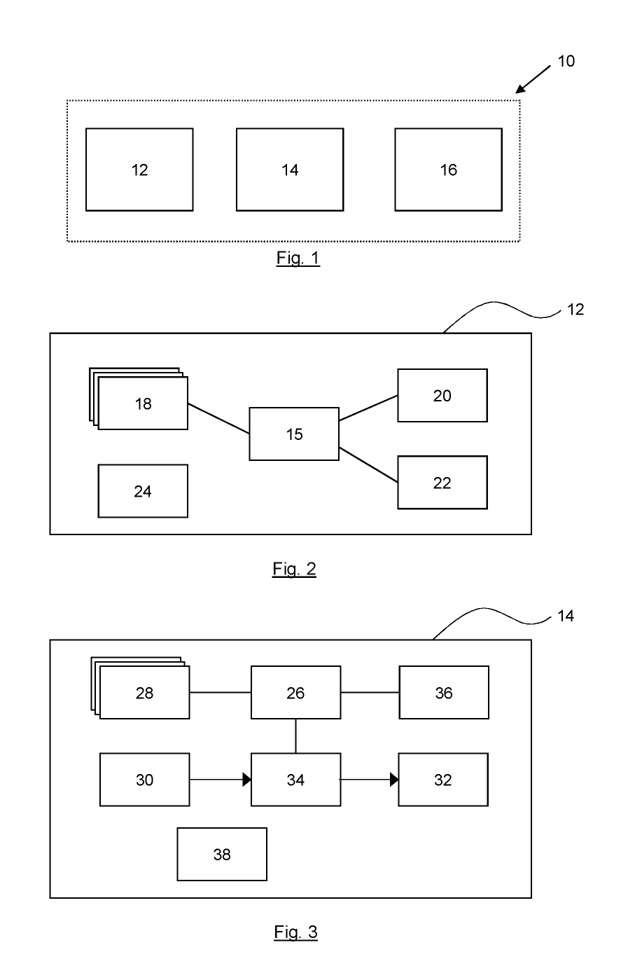

As illustrated figuratively in FIG. 2, the glucometer 12 comprises a processor 15, one or more memory modules 18 (which may comprise volatile and/or non-volatile memory), a media reader 20, a transmitter 22, and a power source 24. In addition, it may optionally comprise other elements (not illustrated), such as an external memory reader, a visual display such as an LCD or LED screen or LEDs, one or more ports configured for connection to a data cable, etc.

The media reader 20 is configured to facilitate analyzing a blood sample, for example disposed on a test media (not illustrated), such as a test strip, disc, drum, cartridge, or any other suitable medium. It may be designed so as to facilitate detecting the glucose level in the blood sample using any suitable method. For example, in an electrochemical method, the blood sample reacts with one or more chemicals impregnated on the test medium. The amount of products of the reaction is proportional to the glucose level in the blood, and can be measured electrically by the media reader 20. Alternatively, the media reader 20 may operate using a coulometric or amperometric method, as is known in the art.

In addition, the media reader 20 may be configured to read information encoded on the test medium, including, but not limited to, calibration information, information regarding the make and/or model of the test medium, and information regarding the manufacturing of the test medium (such as batch number, manufacture date, expiration date, etc.).

Typically, the media is provided in packages having a known number of individual media therein.

The transmitter 22 is configured to transmit information regarding the results of the analysis to the remote computing device 16. For example, the glucometer 12 may be configured to transmit the information over a one-way communication channel, such as an audio-based channel, e.g., as described in co-pending U.S. application Ser. No. 14/071,744, and/or using a visual display. According to other examples, the glucometer 12 may be configured to transmit the information over a two-way communication channel, including, but not limited to, BLUETOOTH (i.e., one or more wireless technologies for exchanging data over short distances using short-wavelength radio transmissions in the ISM band from 2400-2480 MHz as per the standards defined by the Bluetooth Special Interest Group), NEAR FIELD COMMUNICATION (i.e., one or more technologies for smartphones and similar devices to establish radio communication with each other by touching them together or bringing them into close proximity, for example based on standards including, but not limited to, ISO/IES 18092 and those defined by the NFC Forum), WI-FI (i.e., one or more wireless local area network products that are based on the Institute of Electrical and Electonic Engineers' 802.11 standards), ZIGBEE (i.e., one or more of a suite of high level communication protocols used to create personal area networks built from small, low-power digital radios based on the Institute of Electrical and Electonic Engineers' 802.15 standard), and/or other suitable protocols.

According to any of the above examples, the transmitter 22 comprises the necessary hardware to facilitate transmission. For example, in the case wherein the glucometer 12 is configured to transmit information over an audio-based channel, the transmitter 22 comprises a suitable speaker. The glucometer 12 may optionally comprise a visual display, e.g., an LCD, LED, or other suitable screen or display, for example in a case wherein it is configured to transmit information using a visual display.

The transmitter 22 may be configured to transmit information over a wired and/or wireless indication channel, mutatis mutandis.

The processor 16 is configured to direct operation of the elements of the glucometer 12. As such, it may be configured to facilitate analysis of the blood sample, encode information for transmitting by the transmitter 22, direct operation of the transmitter, etc. In performing these tasks, it may utilize the one or more memory modules 18 for short-term storage of information.

The insulin pump 14 is any device configured to administer a dose of insulin to a user. As illustrated figuratively in FIG. 3, it comprises a processor 26, one or more memory modules 28 (which may comprise volatile and/or non-volatile memory), a reservoir 30 configured for containing therein insulin, an infusion set interface 32 configured for attachment thereto of an infusion set, a pump 34 configured to move the insulin from the reservoir to the infusion set interface by mechanical means, a transceiver 36, and a power source 38.

As noted above, the reservoir 30 is configured for containing therein insulin. It may be a refillable insulin reservoir, either permanently mounted within or detachable from/re-attachable to the insulin pump 14. Alternatively, it may be configured for receiving therein insulin a single time, for example by the manufacturer, and for being disposed of by the user when the insulin supply therein is depleted. The processor 26 may be configured for determining the amount of insulin remaining in the reservoir 30, and to facilitate transmission of information regarding to amount remaining via the transceiver 36.

The transceiver 36 is configured to transmit and receive electronic communications. For example, the insulin pump 14 may be configured to transmit the information over a two-way communication channel, including, but not limited to, BLUETOOTH (i.e., one or more wireless technologies for exchanging data over short distances using short-wavelength radio transmissions in the ISM band from 2400-2480 MHz as per the standards defined by the Bluetooth Special Interest Group), NEAR FIELD COMMUNICATION (i.e., one or more technologies for smartphones and similar devices to establish radio communication with each other by touching them together or bringing them into close proximity, for example based on standards including, but not limited to, ISO/IES 18092 and those defined by the NFC Forum), WI-FI (i.e., one or more wireless local area network products that are based on the Institute of Electrical and Electonic Engineers' 802.11 standards), ZIGBEE (i.e., one or more of a suite of high level communication protocols used to create personal area networks built from small, low-power digital radios based on the Institute of Electrical and Electonic Engineers' 802.15 standard), and/or other suitable protocols.

According to any of the above examples, the transceiver 36 comprises the necessary hardware to facilitate transmission and receiving.

The remote computing device 16 is any suitable device configured to receive information transmitted by the transmitter 22 of the glucometer 12, transmit and receive information to the transceiver 36 of the insulin pump 14, execute a program, display information to a user, and receive commands from a user. It may also be configured to communicate with an external network, for example a public network such as the Internet, a POTS network, an ISDN network, cellular telephone system, and/or a VoIP system. As such, it may be any computing device, such as a mobile phone built on a mobile operating system (also referred to as a “smartphone”), a tablet computer, or any other suitable device. In particular, the remote computing device 16 is configured for installation thereon of third-party software.

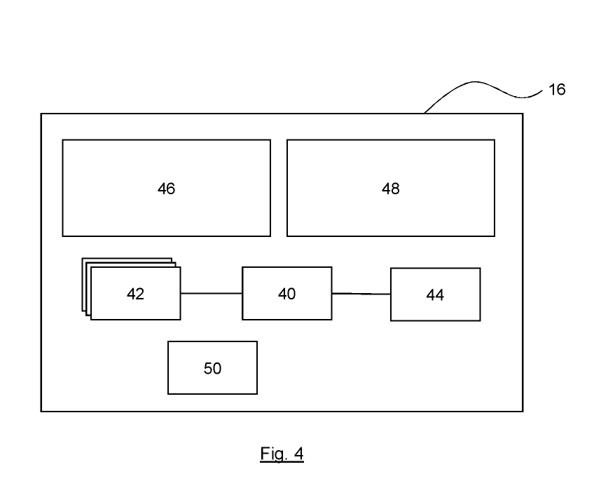

As illustrated schematically in the FIG. 4, the remote computing device 16 comprises a processor 40, one or more memory modules 42 (which may comprise volatile and/or non-volatile memory), one or more transceivers 44, a user-input interface 46, a user-output interface 48, and a power source 50. The user-input interface 46 and user-output interface 48 may be part of the same element, e.g., a touch-screen may constitute both.

The processor 40 is configured to direct operation of the remote computing device 16. Inter alia, it is configured to execute software stored in the memory modules 42. In addition, the processor 26 may be configured to facilitate updating software stored in the memory modules 28, for example by downloading updated software from a remote server via the Internet.

The transceivers 44 are each configured to transmit and receive electronic communications. For example, the transceivers 44 may comprise a modem and/or devices configured to transmit information over a two-way communication channel, including, but not limited to, one or more of BLUETOOTH (i.e., one or more wireless technologies for exchanging data over short distances using short-wavelength radio transmissions in the ISM band from 2400-2480 MHz as per the standards defined by the Bluetooth Special Interest Group), NEAR FIELD COMMUNICATION (i.e., one or more technologies for smartphones and similar devices to establish radio communication with each other by touching them together or bringing them into close proximity, for example based on standards including, but not limited to, ISO/IES 18092 and those defined by the NFC Forum), WI-FI (i.e., one or more wireless local area network products that are based on the Institute of Electrical and Electonic Engineers' 802.11 standards), ZIGBEE (i.e., one or more of a suite of high level communication protocols used to create personal area networks built from small, low-power digital radios based on the Institute of Electrical and Electonic Engineers' 802.15 standard), and/or other suitable protocols.

According to any of the above examples, the transceiver 44 comprises the necessary hardware to facilitate transmission and receiving.

When constituting part of the system 10, the remote computing device 16 is loaded with a software application which is configured to function with the glucometer 12. For example, the information transmitted by the glucometer 12 may contain raw data obtained by the media reader 20, which the software application is configured to interpret and provide a useful value based thereon. It may further be provided to track glucose levels over time, communicate with one or more outside servers, etc.

In addition, the remote computing device 16, when constituting part of the system 10, is loaded with a software application which is configured to direct operation of the insulin pump 14. It will be appreciated that in order to safely direct operation of the insulin pump 14, the software application is configured to both issue commands to and receive information from the insulin pump.

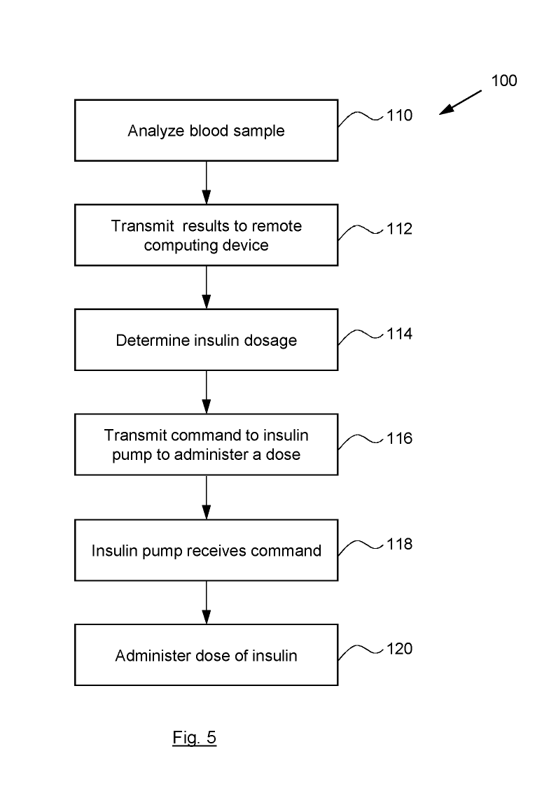

As illustrated in FIG. 5, the system 10, e.g., by running appropriate software (either installed on one of its constituent devices, or as complementary software on two or more thereof), is configured to execute a method, which is generally indicated at 100, for facilitating the remote computing device 16 to direct operation of the insulin pump 14.

In step 110, a user utilizes the glucometer 12 to analyze a blood sample to measure its glucose level. This may be performed according to any suitable method known in the art, for example as described above with reference to FIG. 2.

In step 112, the glucometer 12 transmits information to the remote computing device 16 regarding the results of the blood analysis performed in step 110.

In step 114, the remote computing device 16 determines an insulin dosage to be administered to the user, based on the information transmitted to it by the glucometer 12 in step 112. In making the determination, the remote computing device 16 may perform a calculation based on relevant factors. Alternatively, the remote computing device 16 may be configured to retrieve dosage information from one or more tables, preloaded thereon, containing pre-calculated insulin dosages based on one or more relevant factors.

In step 116, the remote computing device 16 transmits a command to the insulin pump 14 to administer a dose of insulin to the user based on the dosage determined in step 114. The command to administer a dose of insulin may be transmitted via one of the transceivers 44 of the remote computing device 16 to the transceiver 36 of the insulin pump 14. According to some examples, this remote computing device 16 is configured to automatically transmit the command when it determines that administering the determined dosage is necessary. According to other examples, the remote computing device 16 is configured to prompt the user to confirm that the command be transmitted to the insulin pump 14, and only transmit the command upon the user's confirmation. According to either of the above examples, the remote computing device 16 may be configured to alert the user to ensure that the insulin pump 14 is ready to administer a dose of insulin, e.g., that it is powered on, properly connected, etc. According to some modifications, the remote computing device 16 is configured to transmit error-detection and/or error-correction information to the insulin pump 14 with the command, for example as is known in the art.

In step 118, the insulin pump 14 receives the command transmitted by the remote computing device 16. This step may further comprise verification of the command by the insulin pump 14.

According to some examples, the system 10 is configured to verify that the intended command was received by the insulin pump. Upon receiving the command, the insulin pump 14 transmits a message to the remote computing device 16, containing relevant information from the command it received. The remote computing device 16 verifies that information in the message it received from the insulin pump 14 matches the relevant information in the command that it issues. Upon verifying the match, the remote computing device 16 sends an acknowledgment message to the insulin pump 14, thereby verifying that the command that the insulin pump 14 received contained proper information.

In step 120, the insulin pump 14 administers a dose of insulin to the user based on the command issue to it by the remote computing device 16. According to some examples, this step requires that a user activates a mechanism, such as a button, knob, switch, etc., of the insulin pump 14 before administration of the insulin commences. This step may further comprise the insulin pump 14 communicating to the remote computing device 16 that the dose was successfully administered.

The system 10 may be configured to determine the quantity of insulin remaining in the reservoir 30 and to initiate an ordering procedure once the level of insulin reaches or is below a predetermined threshold. In determining the threshold, the system 10 may take into account not only the amount of insulin contained in the reservoir 30, but also the user's total supply of insulin.

If the system 10 determines that the threshold value has been passed, it initiates an ordering procedure. The ordering procedure may be any procedure which is designed to facilitate or otherwise directly contribute toward additional insulin being ordered.

According to some examples, the ordering procedure comprises the remote computing device 16 ordering a new supply of insulin via the Internet from an Internet-based merchant. This may be performed by the remote computing device 16 automatically. Accordingly, the remote computing device 16 and/or Internet-based merchant stores relevant information, e.g., shipping address, product to be ordered, etc. According to some modifications, the remote computing device 16 and/or Internet-based merchant may further store billing data, such as credit card information, billing address, etc. The remote computing device 16 may be configured to survey several ordering options for the best value. For example, it may check several Internet-based merchants for the best price for a particular product, it may check for the best unit price among several equivalent products, either at a single or at several Internet-based merchants, etc., and order from the one which provides the best value. In addition, in may be configured to take a preselected action if it identifies an unusually good value, for example as preset by the user. The preselected action may be one or more of, but is not limited to, ordering an increased quantity of insulin and/or alerting the user that it identified the value. The user may preset what he considers to be a good value, for example more than a certain percentage lower per unit volume of insulin than past purchases. The past purchases may be, e.g., an average or absolute best value of a predetermined number of previous purchases, an average or absolute best value of purchases made over a preselected amount of time, etc.

According to other examples, the ordering may be a manual process, wherein the ordering procedure comprises alerting the user that the system 10 has determined that an order should be placed for additional insulin. The remote computing device 16 may optionally present information and/or direction to facilitate the ordering by the user. For example, it may present on its user-output interface 48 one or more links, each redirecting a user to an Internet-based merchant which sells one or more packages of suitable insulin.

According to further examples, some parts of the ordering may be automatic, as described above, with one or more manual steps. According to some modifications, the ordering procedure may comprise the remote computing device 16 automatically retrieving a website via which the user can purchase a package of insulin. According to other modifications, the ordering procedure may comprise the remote computing device 16 retrieving all information necessary to place an order, e.g., product, price, merchant, estimate of shipping time, shipping address, billing address, billing information, etc., and presenting to the user for his approval, upon which the remote computing devices places the order accordingly.

According to any of the above examples, the system 10 may be configured to conduct the ordering procedure in accordance with a prescription issued by the user's physician.

Those skilled in the art to which this invention pertains will readily appreciate that numerous changes, variations and modifications can be made without departing from the scope of the invention mutatis mutandis.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

What is claimed is:

1. A method of administering insulin to a user, the method comprising:
providing a system comprising a glucometer configured to analyze a blood sample, a remote computing device separate from said glucometer, and an insulin pump separate from said remote computing device and glucometer;
analyzing, by said glucometer, a blood sample from a user, and communicating said results to said remote computing device;
determining, by said remote computing device and based on said results, an insulin dosage to be administered;
communicating, by said remote computing device, a command to said insulin pump to administer a dose of insulin based on the determined dosage; and
administering, by said insulin pump, said dose.

2. The method according to claim 1, wherein said determining comprises calculating a dosage to be administered.

3. The method according to claim 1, wherein said determining comprises retrieving dosage information from one or more tables preloaded on the remote computing device.

4. The method according to claim 1, wherein said remote computing device is configured to automatically communicate said command.

5. The method according to claim 1, wherein said remote computing device is configured to communicate said command upon confirmation by a user.

6. The method according to claim 1, wherein said communicating further comprises transmitting one or more of error-detection and error-correction information.

7. The method according to claim 1, further comprising said system verifying that an intended command was received by the insulin pump.

8. The method according to claim 1, further comprising, prior to said administering, a user activating a mechanism on the insulin pump.

9. A system configured to administer insulin to a user, said system comprising a glucometer configured to analyze a blood sample, a remote computing device separate from said glucometer, and an insulin pump separate from said remote computing device and glucometer, wherein said remote computing device is configured to receive communication from said glucometer regarding results of a blood analysis, determine, based on said results, an insulin dosage to be administered, and communicate a command to said insulin pump to administer a dose of insulin based on the determined dosage, said insulin pump being configured to administer said dose.

10. The system according to claim 9, said remote computing device being configured to determining the insulin dose by calculating a dosage to be administered.

11. The system according to claim 9, said remote computing device being configured to determining the insulin dose by retrieving dosage information from one or more tables preloaded on the remote computing device.

12. The system according to claim 9, wherein said remote computing device is configured to automatically communicate said command.

13. The system according to claim 9, wherein said remote computing device is configured to communicate said command upon confirmation by a user.

14. The system according to claim 9, wherein said remote computing device is further configured to transmitting one or more of error-detection and error-correction information to said insulin pump.

15. The system according to claim 9, being configured to verify that an intended command was received by the insulin pump.

16. The system according to claim 9, wherein said insulin pump is configured to administer said dose upon activation of a mechanism thereof.

17. A device configured to administer an insulin dose to a user, the device comprising a pump configured for the administering, a communications interface configured to receive instructions from a remote computing device, and a processor, said processor being configured to:
receive at least one instruction via said communications interface; and
operate said pump to administer an insulin dose based on said instruction.
